(19) United States
(12) Patent Application Publication
(10) Pub. No.: US 2017/0176461 A1
(43) Pub. Date: Jun. 22, 2017

(54) SYSTEMS AND METHODS FOR QUANTIFICATION OF CREATININE USING A CREATININE-PROTEIN CONJUGATE

(71) Applicant: Polymer Technology Systems, Inc., Indianapolis, IN (US)

(72) Inventors: Aniruddha Patwardhan, Fishers, IN (US); Ryan Jesswein, Indianapolis, IN (US); Keith Moskowitz, Indianapolis, IN (US)

(21) Appl. No.: 15/386,958

(22) Filed: Dec. 21, 2016

Related U.S. Application Data
(60) Provisional application No. 62/271,223, filed on Dec. 22, 2015.

(51) Int. Cl. G01N 33/70; G01N 33/58; G01N 33/543

(52) U.S. Cl. CPC G01N 33/70; G01N 33/54313; G01N 33/582; G01N 33/54386

(57) ABSTRACT

A system for determining a level of creatinine in a sample includes a test strip system configured to receive a sample, the test strip system including a first lateral flow test strip and a meter configured to receive the test strip, wherein the meter is configured to read the test strip and detect a level of creatinine, wherein the first lateral flow test strip includes microparticles combined with a creatinine antibody.

### CROSS REFERENCE TO RELATED APPLICATION

This application claims the benefit of U.S. Provisional Patent Application 62/271,223 filed Dec. 22, 2015, and hereby incorporated by reference to the same extent as though fully disclosed herein.

### BACKGROUND

Creatinine (C₄H₉O₂N₃ or α-methyl guanidine-acetic acid) is a compound present in vertebrate muscle tissue, principally as phosphocreatine. Creatinine is synthesized primarily in the liver and also in the pancreas and the kidneys. Creatinine helps produce energy needed to contract muscles, and it is produced at a relatively constant rate. Creatinine is eventually spontaneously degraded into creatinine by muscle and is released into the blood. It then is excreted by the kidneys and removed from the body by glomerular filtration.

The amount of creatinine produced is relatively stable in a given person. A serum creatinine level, therefore, is determined by the rate it is being removed, which is roughly a measure of kidney function. If kidney function falls, the serum creatinine level will rise. Thus, blood levels of creatinine are a good measure of renal function. Usually, increased creatinine levels do not appear unless significant renal impairment exists.

According to the American Diabetes Association (ADA), 20% to 30% of patients with diabetes develop diabetic kidney disease (nephropathy). Further, some authorities recommend measurement of serum creatinine levels in non-diabetic patients to screen for renal dysfunction because of increasing evidence that dietary protein restriction and use of angiotensin-converting enzyme (ACE) inhibitors can retard progression once renal insufficiency develops. Thus, the need for creatinine testing as a measure of kidney function is well established.

### SUMMARY

In one embodiment, a system for determining a level of creatinine in a sample includes a test strip system configured to receive a sample, the test strip system including a first lateral flow test strip and a meter configured to receive the test strip, wherein the meter is configured to read the test strip and detect a level of creatinine, wherein the first lateral flow test strip includes microparticles combined with a creatinine antibody. Optionally, the first lateral flow test strip includes compounds to bind with the microparticles combined with the creatinine antibody; the compounds are a creatinine-protein conjugate. Alternatively, the creatinine-protein conjugate is a creatinine BSA conjugate. In one configuration, the test strip system includes a sample pad oriented in line with an opening in a cartridge, the cartridge holding the sample pad and the first lateral flow test strip. In another configuration, the microparticles are fluorescent. Alternatively, the microparticles have reflective properties. In one alternative, the microparticles have properties that provide for the absorption of light. Optionally, the system further includes a second lateral flow test strip, the second lateral flow test strip in communication with the sample pad, wherein the meter is configured to read the second lateral flow test strip and provide an indication of a second level of creatinine in the sample. Alternatively, the meter provides an estimation of a level of creatinine based on the first level of creatinine and the second level of the creatinine.

In one embodiment, a test strip system for determining a level of creatinine in a sample includes a first lateral flow test strip, wherein the first lateral flow test strip includes microparticles combined with a creatinine antibody. Optionally, the first lateral flow test strip includes compounds to bind with the microparticles combined with the creatinine antibody; the compounds are a creatinine-protein conjugate. Alternatively, the creatinine-protein conjugate is a creatinine BSA conjugate. Alternatively, the system includes a sample pad oriented in line with an opening in a cartridge, the cartridge holding the sample pad and the first lateral flow test strip. Optionally, the microparticles are fluorescent. In one alternative, the microparticles have reflective properties. In another alternative, the microparticles have properties that provide for the absorption of light. In one alternative, the system includes a second lateral flow test strip, the second lateral flow test strip in communication with the sample pad, wherein the meter is configured to read the second lateral flow test strip and provide an indication of a second level of creatinine in the sample.

In one embodiment, a method of determining a level of creatinine in a sample includes providing a first lateral flow test strip, wherein the first lateral flow test strip includes microparticles combined with a creatinine antibody and providing a meter configured to receive the test strip, wherein the meter is configured to read the first lateral flow test strip and detect a level of creatinine. The method further includes placing a sample on the first lateral flow test strip; laterally flowing the sample of the test strip; and reading the test strip with the meter. Optionally, the first lateral flow test strip includes compounds to bind with the microparticles combined with the creatinine antibody; the compounds are a creatinine-protein conjugate. Alternatively, the creatinine-protein conjugate is a creatinine BSA conjugate or any other carrier proteins (like Human serum albumin, Keyhole limpet hemocyanin (KLH), ovalbumin, etc.). Alternatively, the test strip system includes a sample pad oriented in line with an opening in a cartridge, the cartridge holding the sample pad and the first lateral flow test strip. Optionally, the microparticles are fluorescent. In one configuration, the microparticles have reflective properties. Alternatively, the microparticles have properties that provide for the absorption of light. Optionally, the method further includes providing a second lateral flow test strip, the second lateral flow test strip in communication with the sample pad and reading the second lateral flow test strip, and providing an indication of a second level of creatinine in the sample. Alternatively, the method further includes providing an estimation of a level of creatinine based on the first level of creatinine and the second level of the creatinine.

### BRIEF DESCRIPTION OF THE DRAWINGS

FIG. 1 shows one embodiment of the components of the lateral flow strip architecture for use with a lateral flow creatinine assay;

FIG. 2 shows an exemplary pathway whereby creatinine may be coupled directly to the BSA or any other carrier proteins (like Human serum albumin, Keyhole limpet hemocyanin (KLH), ovalbumin, etc.);

FIG. 3a shows an embodiment of a scheme (or reaction pathway) where the thioctic acid is coupled with creatinine using N-Hydroxy Succinamide (NHS)/1-Ethyl-3-(3-dimethylaminopropyl) carbodiimide (EDAC) followed by conjugation to a modified protein (using 6-Maleimidohexanoic acid N-Hydroxy succinic acid) to give a final product;

FIG. 3b shows an embodiment of a scheme (or reaction pathway) where the 2-iminothiolane is reacted with creatinine to give a free sulfhydryl product “A”, which in turn is reacted with a modified protein “B” (using 6-Maleimidohexanoic acid N-Hydroxy succinic acid) to give a final product;

FIG. 4 shows an exemplary lateral flow test strip for use in the testing of creatinine;

FIG. 5 shows a graph of reflectance vs. concentration of creatinine;

FIG. 6a is an example of a gel that shows the the conjugate prepared via the Trauts reagent process alongside the free BSA protein when stained with Ponceau S to show the position of the bands on a gel; and

FIG. 6b is an example of a western blot in which the stain on the membrane from FIG. 6a is washed and the membrane is subsequently exposed to primary anti-creatinine antibody, donkey anti-sheep IgG HRP conjugate secondary, and TMB blotting solution.

### DETAILED DESCRIPTION

Certain terminology is used herein for convenience only and is not to be taken as a limitation on the embodiments of the systems and methods for the detection and quantification of creatinine using a creatinine-protein conjugate. In the drawings, the same reference letters are employed for designating the same elements throughout the several figures.

In many embodiments of the systems and method described herein, a novel creatinine-protein conjugate is provided. This conjugate may be used in an assay as a component to quantify the levels of creatinine in whole blood. Embodiments of this system have numerous advantages, including, but not limited to:
1. Embodiments provide for the rapid preparation of a conjugate in the laboratory without using extensive organic chemistry techniques.
2. Embodiments of methods also utilize dialysis techniques and eliminate cumbersome purification steps using column chromatography.
3. Embodiments of methods also enable rapid scale-up of the conjugate, thus offering versatility.

Creatinine is a breakdown product of creatinine phosphate in muscle and usually is produced at a constant rate by the body. It is an important indicator of renal health because it is an easily measured analyte of muscle metabolism that is excreted unchanged by the kidneys.

Creatinine is removed from the blood by the kidneys, primarily by glomerular filtration. If the filtration in the kidney is deficient, creatinine blood levels rise. Therefore, creatinine levels in blood and urine may be used to calculate the creatinine clearance (CrCl), which correlates with the glomerular filtration rate (GFR). Blood creatinine levels may also be used alone to calculate the estimated GFR (eGFR). The GFR is clinically important because it is a measurement of renal function.

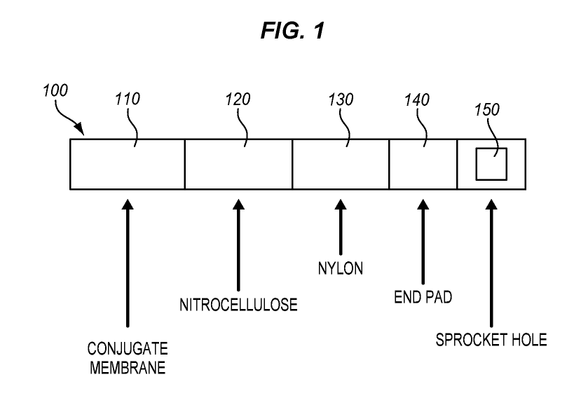

Many embodiments include the development of a point-of-care test to quantify the levels of creatinine in whole blood. A lateral flow platform has been chosen in many configurations to develop the creatinine test. In this platform, a lateral flow strip (as shown in FIG. 1) usually consists of membranes which are coated with polystyrene particles coated with antibodies on the conjugate membrane portion while the creatinine antigen (creatinine-BSA conjugate) is coated on the nylon membrane portion of the lateral flow membrane.

In one embodiment, the point-of-care testing device employs a lateral flow methodology. In this lateral flow method, the conjugate membrane, nitrocellulose membrane, and nylon membrane are layered in such a way as to obtain easy plasma/fluid flow which enables the analytes to be captured on the membranes in different zones as shown in FIG. 1. FIG. 1 shows one embodiment of the components of the lateral flow strip architecture. FIG. 1 shows a lateral flow test strip 100. The lateral flow test strip 100 includes a conjugate membrane 110, a nitrocellulose membrane 120, a nylon membrane 130, an end pad 140, and a sprocket hole 150 for mounting the lateral flow test strip on a test strip holder, cartridge, or cassette.

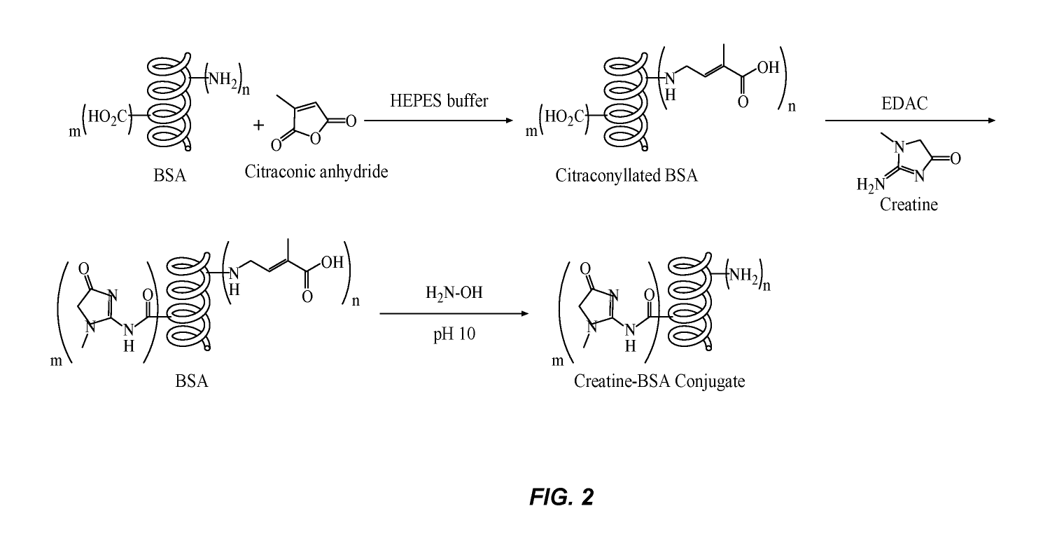

In many embodiments, a key aspect is to develop a creatinine-protein (like Bovine Serum Albumin {BSA} or any other carrier proteins (like Human serum albumin, Keyhole limpet hemocyanin (KLH), ovalbumin, etc.) conjugate that will be used to stripe on the nylon portion of the lateral flow membrane. For preparing the creatinine-protein conjugate, BSA (Bovine serum albumin) was chosen as the protein because it would not interfere in the lateral flow methodology. Alternative conjugates using different proteins may be used, however, BSA is typically the most cost effective. Three methods were used to prepare the creatinine-BSA conjugate. FIG. 2 shows that the creatinine may be coupled directly to the BSA protein. In this approach, a protection-deprotection strategy was used. The amines moiety of the amino acid groups on BSA were citraconyllated using citraconic anhydride. After dialyzing out the unreacted citraconic anhydride, the citraconyllated BSA's carboxylic groups were coupled with creatinine's amino group using ethyl dimethylamino carbodiimide (EDAC) in phosphate buffered saline (PBS) buffer. In alternative embodiments, different buffers may be used in the reaction. The resulting creatinine-BSA-citraconic acid product then was dialyzed to remove unreacted creatinine and subjected to deprotection of the citraconic acid moiety using hydroxylamine at pH 10. The pH suggested for this process may be different in alternative embodiments, but it is thought that in many scenarios a pH greater than 8 is necessary. After extensive dialysis in PBS buffer, the creatinine-BSA conjugate was obtained. If an alternative buffer is used, then the dialysis step with also utilize the alternative buffer. Therefore, in some embodiments a method of coupling creatinine to BSA protein may include the above steps.

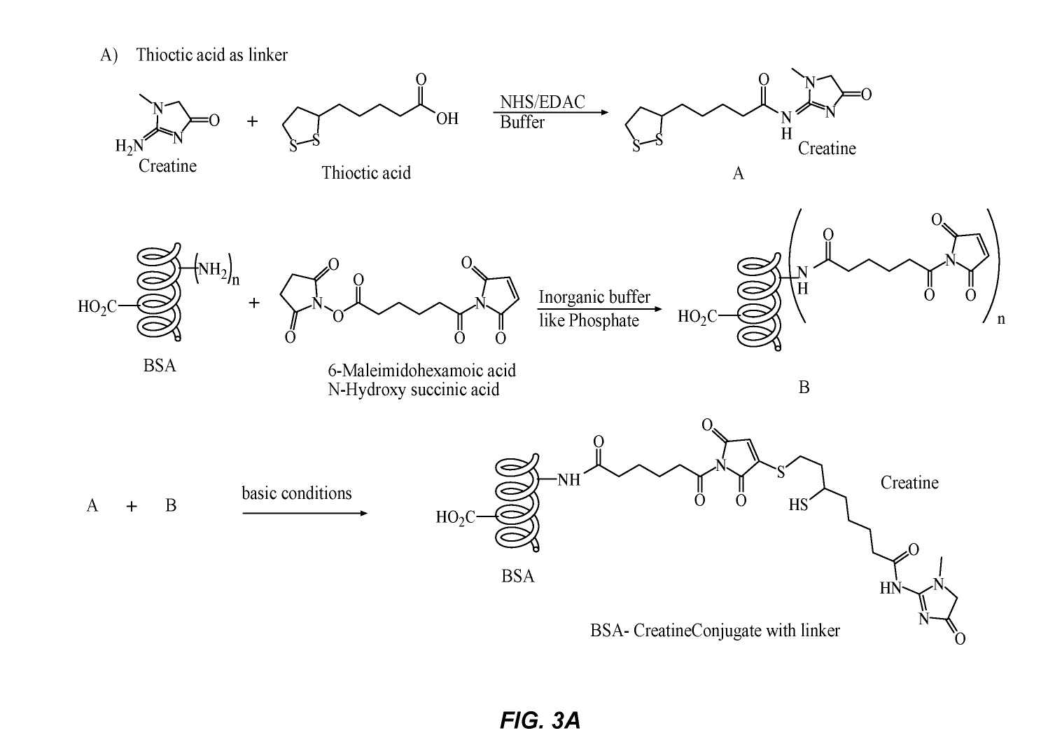

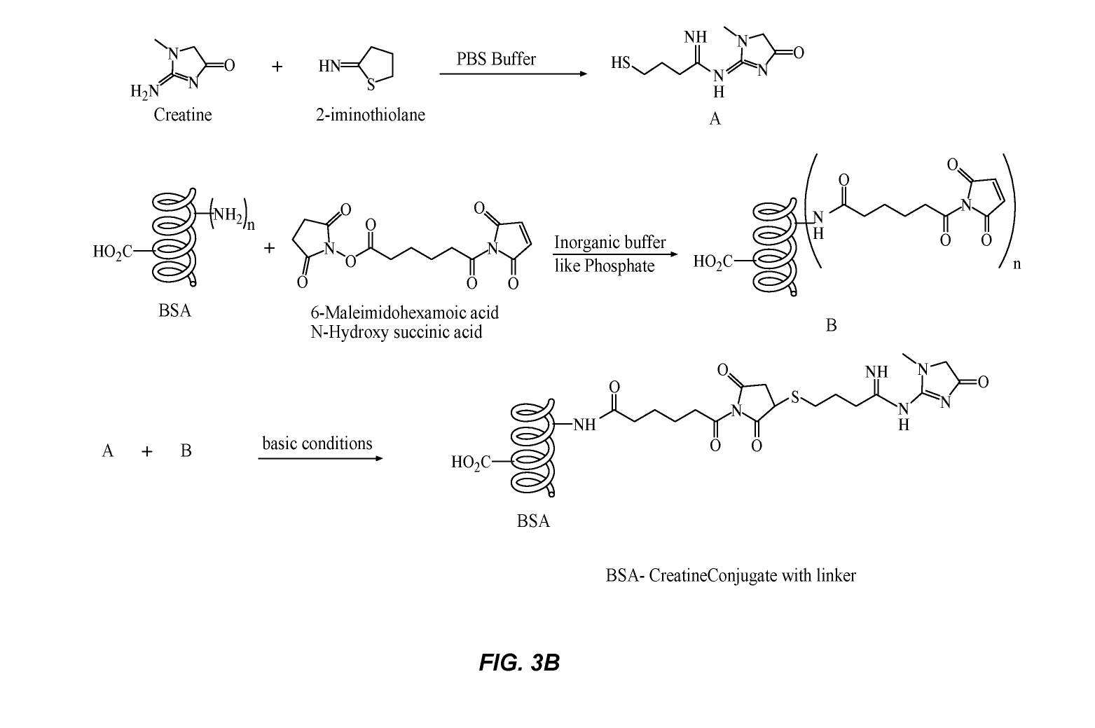

Another approach is to use a linker where the creatinine is attached to the BSA protein. Two reagents used are the thioctic acid and the 2-iminothiolane along with the bifunctional linker 6-maleimidohexanoic acid N-hydroxy succinic acid. FIG. 3a shows an embodiment of a scheme (or reaction pathway) where the thioctic acid is coupled with creatinine using EDAC to give product “A.” This is performed in a NHS/EDAC buffer, however other buffers are possible. BSA is reacted with 6-maleimidohexanoic acid N-hydroxy succinc acid to yield a protein with a derivatized linker “B” that contains a maleimide moiety which can undergo Michael addition reaction when a nucleophile like a thiol group is added; in this case, the compound “A.” The creation of product “B” is done in an inorganic buffer like Phosphate although other buffers are possible. The products “A” and “B” are preferably performed in basic conditions. After extensive dialysis, the final product (a creatinine-protein conjugate) is formed. Therefore, one inventive concept contained herein is a method including the above steps using a linker to attach the creatinine to the BSA protein

FIG. 3b shows an embodiment of a scheme (or reaction pathway) where the 2-iminothiolane is reacted with creatinine to give a free sulfhydryl product “A.” BSA is reacted with 6-maleimidohexanoic acid N-hydroxy succinc acid to yield a protein with a derivatized linker “B.” This is done in an inorganic buffer like Phosphate although other buffers are possible. This protein adduct “B” then is reacted overnight with the free thiol reactant “A” to yield the final product after extensive dialysis. This is done in basic conditions. Therefore, one inventive concept contained herein is a method including the above steps

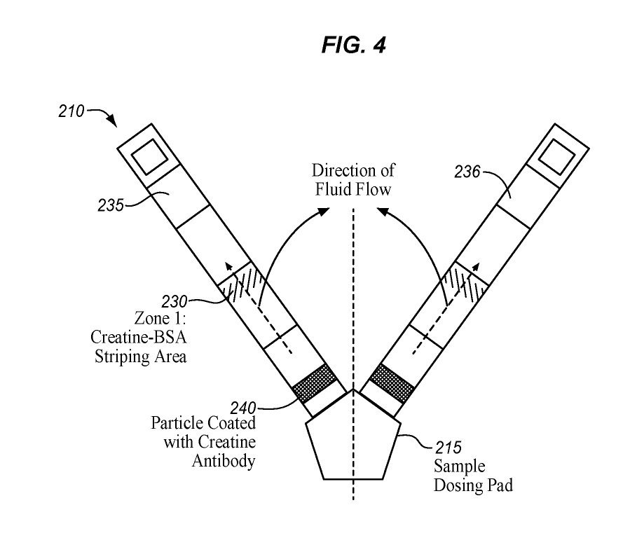

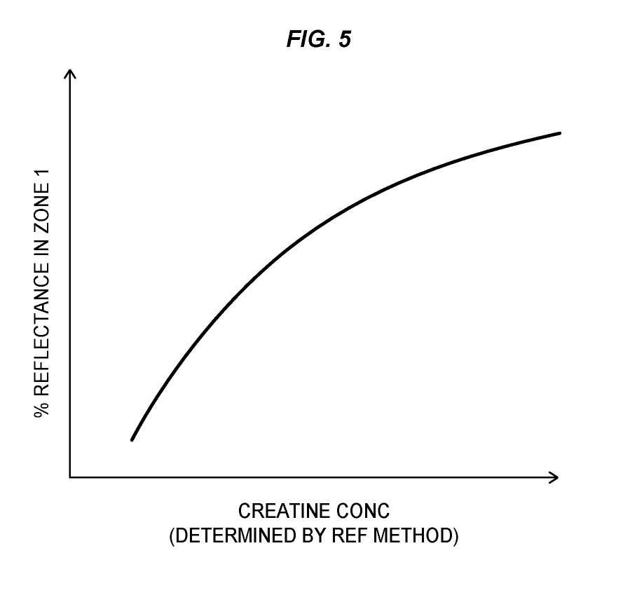

In one embodiment of a creatinine lateral flow assay, a creatinine protein-conjugate described above is used. In many embodiments, a creatinine lateral flow assay 210 may be created. The conjugate(s) described above will be coated on the area in zone 1 230 as shown in FIG. 4. The blue dyed particles will be coated with particle coated anti-creatinine antibodies at stripe 240.The system may include two arms 235, 236 and may include a sample dosing pad 215. It is certainly possible to only include a single arm; however, the redundancy may be used to prevent inaccuracies in measurements. When a sample containing creatinine is dosed, the antibodies on the particles will bind the creatinine in the sample. As the particles flow up the lateral flow membrane, the particles containing anti-creatinine antibodies (that are not bound with free creatinine) will bind to the creatinine on the conjugate, resulting in a response. When a very low concentration of creatinine is present, the particles will bind the conjugate striped in zone 1 230 the most. As the creatinine concentration in the sample increases, the particles with creatinine antibodies will bind the free creatinine and, as a result, the particles will not bind the conjugate in zone 1 230, eading to a higher reflectance. If the quantity of the creatinine is titrated from low to high, a dose response as shown in FIG. 5 is obtained and thus enables the quantification of creatinine in the sample.

In many embodiments, a premix step may be included. Typically, a sample is exposed to a premix step with a buffer solution. After a premix, the sample and buffer solution are applied to a test strip. The test strip includes an antibody-microparticle zone and a capture zone for capturing antibodies that have not reacted with the sample. Typically, after the lateral flow of the sample, the flow of the microparticles is measured using an optical meter. Various lateral flow membranes may be utilized.

In one example, conjugates have been prepared and show creatinine has been attached on the protein when tested on a colorimetric creatinine assay on the Integra. The amount of creatinine covalently attached to the BSA protein conjugates were determined digesting the conjugate using pepsin. This digestion step was necessary to “liberate” the amino acids areas or domains of the BSA protein where the creatinine is covalently attached. The liberated amino acids with covalently attached creatinine reacts easily in a commercially available creatinine assay on clinical analyzer platforms. If one does not digest the conjugate, the amount of creatinine covalently bound on the protein, the creatinine values obtained are very low. This is easily explained by the fact that the large BSA molecule severely hindered the reactivity of the creatinine molecule in an enzymatic reaction. The table below shows the amount of BSA, creatinine pre- and post-pepsin digestion, as well as the creatinine-to-BSA ratio. This ratio is important, as it will be used to “dial in” in a correct amount conjugate that will offer a constant creatinine-to-BSA ratio during the assay development.

| | | | Creatinine | |
|---|---|---|---|---|
| | BSA | Creatinine | Reference | |
| Conjugate | Protein | Reference | Analyzer | Creatinine- |
| Reaction | Assay | Analyzer (Pre- | (Pepsin | to- |
| Scheme | Result | Digestion) | Digestion) | BSA-Ratio |
| Direct Method | 314 mg/dL | 0.25 mg/dL | 4.3 mg/dL | 5.4:1 |
| (FIG. 2) | | | | |
| Thioctic Acid | 105 mg/dL | 0.20 mg/dL | 4.1 mg/dL | 6.2:1 |
| method | | | | |
| (FIG. 3A) | | | | |
| 2-iminothiolane | 115 mg/dL | 0.60 mg/dL | 5.3 mg/dL | 2.8:1 |
| method | | | | |
| (FIG. 3B) | | | | |

The conjugates then were subjected to Western Blot techniques to determine if the protein had been dimerized during the conjugate preparation method, as well as to detect qualitatively if the creatinine molecule had been attached to the protein.

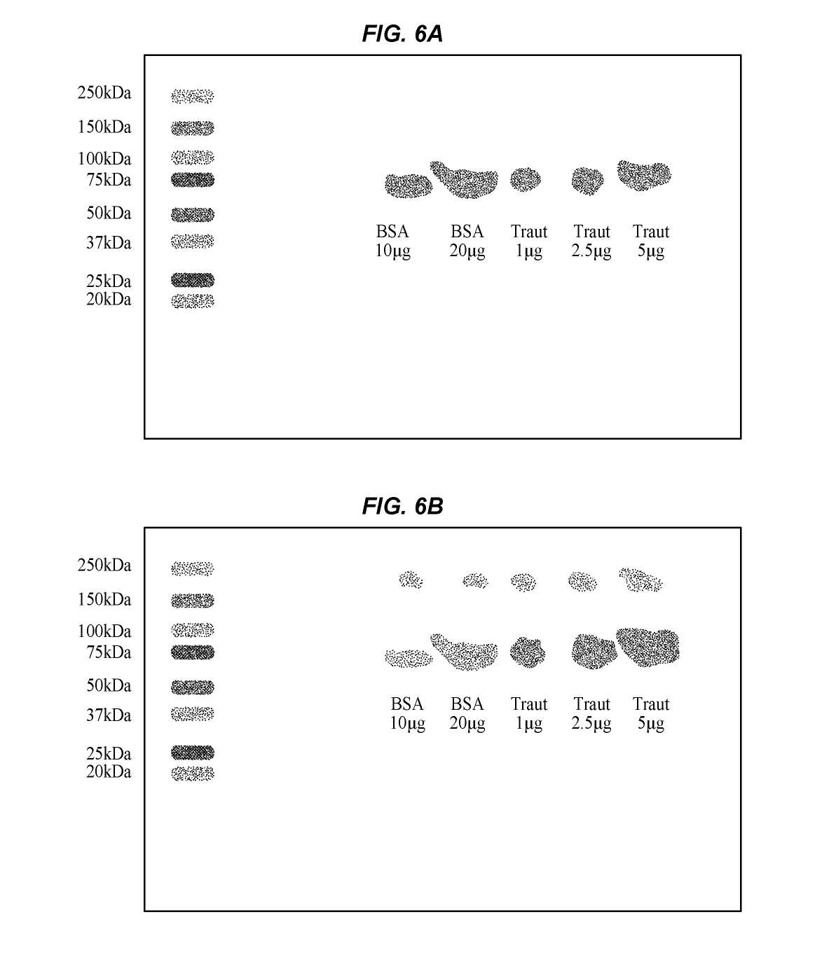

FIG. 6a is an example of a gel that shows the conjugate prepared via the Trauts reagent process alongside the BSA protein when stained with Ponceau S to show the position of the bands on a gel. The molecular ladder ranged from 250-20 kDa which is on the left of the gel. The gel in FIG. 6 shows the relative position (per the molecular markers in the far left side of the gel) of the un-conjugated BSA at 10 μgm and 20 μgm, while the conjugate prepared using the Trauts reagent was loaded on the gel at 1 μgm, 2.5 μgm, and 5 μgm. The image shows that the transfer from the gel to the nitrocellulose paper has occurred efficiently. Importantly, the conjugate had the the molecular weight as that of the BSA demonstrating that the conjugation did not lead to major dimerized product. There is a hint of a dimer at 150 kDa, but that also is observed for native BSA, thus indicating that the BSA source may contain some dimerized BSA. FIG. 6b is an example of a western blot in which the stain on the membrane from FIG. 6a is washed and the membrane is subsequently exposed to primary anti-creatinine antibody, donkey anti-sheep IgG HRP conjugate secondary, and TMB blotting solution. This figure shows the conjugate turned blue indicating the presence of the creatinine on the protein. There is a lack of blue color in the relative position of the un-conjugated BSA.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof.

What is claimed as new and desired to be protected by Letters Patent of the United States is:

1. A system for determining a level of creatinine in a sample, comprising:
a test strip system configured to receive a sample, the test strip system including a first lateral flow test strip; and
a meter configured to receive the test strip, wherein the meter is configured to read the test strip and detect a level of creatinine, wherein the first lateral flow test strip includes microparticles combined with a creatinine antibody.

2. The system of claim 1, wherein the first lateral flow test strip includes compounds to bind with the microparticles combined with the creatinine antibody, wherein the compounds are a creatinine-protein conjugate.

3. The system of claim 2, wherein the creatinine-protein conjugate is a creatinine BSA conjugate.

4. The system of claim 1, wherein the creatinine-protein conjugate includes a carrier protein selected from a group consisting of Human serum albumin, Keyhole limpet hemocyanin (KLH), and ovalbumin.

5. The system of claim 3, wherein the test strip system includes a sample pad oriented in line with an opening in a cartridge, the cartridge holding the sample pad and the first lateral flow test strip.

6. The system of claim 5, wherein the microparticles are fluorescent.

7. The system of claim 5, wherein the microparticles have reflective properties.

8. The system of claim 5, wherein the microparticles have properties that provide for the absorption of light.

9. The system of claim 5, further comprising a second lateral flow test strip, the second lateral flow test strip in communication with the sample pad, wherein the meter is configured to read the second lateral flow test strip and provide an indication of a second level of creatinine in the sample.

10. The system of claim 9, wherein the meter provides an estimation of a level of creatinine based on the first level of creatinine and the second level of creatinine.

11. A test strip system for determining a level of creatinine in a sample, comprising:
a first lateral flow test strip, wherein the first lateral flow test strip includes microparticles combined with a creatinine antibody.

12. The system of claim 11, wherein the first lateral flow test strip includes compounds to bind with the microparticles combined with the creatinine antibody wherein the compounds are a creatinine-protein conjugate.

13. The system of claim 12, wherein the creatinine-protein conjugate is a creatinine BSA conjugate.

14. The system of claim 12, wherein the creatinine-protein conjugate includes a carrier protein selected from a group consisting of Human serum albumin, Keyhole limpet hemocyanin (KLH), and ovalbumin.

15. The system of claim 13, further comprising a sample pad oriented in line with an opening in a cartridge, the cartridge holding the sample pad and the first lateral flow test strip.

16. The system of claim 14, wherein the microparticles are fluorescent.

17. The system of claim 14, wherein the microparticles have reflective properties.

18. The system of claim 14, wherein the microparticles have properties that provide for the absorption of light.

19. The system of claim 14, further comprising a second lateral flow test strip, the second lateral flow test strip in communication with the sample pad, wherein the meter is configured to read the second lateral flow test strip and provide an indication of a second level of creatinine in the sample.

20. A method of determining a level of creatinine in a sample comprising:
providing a first lateral flow test strip wherein the first lateral flow test strip includes microparticles combined with a creatinine antibody;
providing a meter configured to receive the test strip wherein the meter is configured to read the first lateral flow test strip and detect a level of creatinine;
placing a sample on the first lateral flow test strip;
laterally flowing the sample of the test strip; and
reading the test strip with the meter.

21. The method of claim 20, wherein the first lateral flow test strip includes compounds to bind with the microparticles combined with the creatinine antibody wherein the compounds are a creatinine-protein conjugate.

22. The method of claim 21, wherein the creatinine-protein conjugate is a creatinine BSA conjugate.

23. The method of claim 21, wherein the creatinine-protein conjugate includes a carrier protein selected from a group consisting of Human serum albumin, Keyhole limpet hemocyanin (KLH), and ovalbumin.

24. The method of claim 22, wherein the test strip system includes a sample pad oriented in line with an opening in a cartridge, the cartridge holding the sample pad and the first lateral flow test strip.

25. The method of claim 24, wherein the microparticles are fluorescent.

26. The method of claim 24, wherein the microparticles have reflective properties.

27. The method of claim 24, wherein the microparticles have properties that provide for the absorption of light.

28. The method of claim 24, further comprising:
providing a second lateral flow test strip, the second lateral flow test strip in communication with the sample pad; and
reading the second lateral flow test strip and providing an indication of a second level of creatinine in the sample.

29. The method of claim 28, further comprising:
providing an estimation of a level of creatinine based on the first level of creatinine and the second level of creatinine.
